(19) United States
(12) Patent Application Publication
(10) Pub. No.: US 2004/0018599 A1
(43) Pub. Date: Jan. 29, 2004

(54) Enhanced production from almond hulls of products such as inositol and inositol phosphates

(76) Inventor: Israel N. Rabinowitz, Santa Barbara, CA (US)

Correspondence Address: DONALD D. MON, Suite 303, 750 East Green Street, Pasadena, CA, 91101, US

(21) Appl. No.: 10/202,505

(22) Filed: Jul. 24, 2002

(51) Int. Cl. C12P 9/00; C12P 7/02

(52) U.S. Cl. 435/131; 435/155

(57) ABSTRACT

Utilizing almond hulls as the starting material, enhancing the production from them of products such as inositol and inositol phosphates.

### FIELD OF THE INVENTION

From almond hulls as the starting material, processes to enhance the production of inositol, inositol phosphates and other pharmacologically useful products utilizing only biological procedures.

### BACKGROUND OF THE INVENTION

The sweet almond is a relative of other stone-containing fruits such as peaches, apricots, and plums. It shares with them the presence of a center stone (the endocarp) and a surrounding flesh layer (the mesocarp). The flesh portion of all of these fruits, including the almond, is edible and enjoyable. However, there is a major difference how they are used in the cultures consuming them.

The peach, apricot and plum are generally eaten fresh, or harvested fresh and dried for later aqueous reconstitution, or eaten as a dried product. The central stone is discarded. Not so with the almond. Generally its end product is the stone, which is the common almond nut that is so widely used, especially in the United States.

In contrast, the almond fruit is only rarely harvested fresh. Instead it is left on the tree to approach senescence and is left to dry on the tree to a low moisture content. Evidently the biological processes undergone by the fruit as it remains on the tree develop quite different chemical constituents, the advantages of which have been widely unappreciated.

When the crop is harvested for its nut content, the dried remainder of the mesocarp is removed and becomes what is commonly called the “almond hull”. It is regarded as a very low value substance, whose uses are such as for co-generation fuel to be burned in power plants, and as a portion of cattle feed ration.

It has been disregarded that the almond hull has a substantial content of sugars, inositol, phytic acid, and many other potentially useful phytochemicals which are otherwise very difficult and expensive to obtain or produce.

It is an object of this invention to utilize the almond hull as a source of these useful compounds, and to provide processes to produce them inexpensively, using only simple biological and separation procedures to do so.

Interestingly, the relatively large amounts of sugars in the almond hull could be a nuisance in recovering and developing other products. It is an object of this invention instead to utilize the sugars to enhance the production of useful products, at the same time removing them and increasing the harvest of the other products.

### BRIEF DESCRIPTIONS OF THE INVENTION

Dry, clean almond hulls are ground to small particles or a powder and are subjected to counter-current aqueous extraction. When filtered out, there results a mass of fiber of no further interest in this instant invention, and an aqueous extract whose treatment is the subject of this invention.

According to this invention, the aqueous solution has added to it a yeast of the type which consumes sugars such as glucose, fructose and sucrose, and converts the phytic acid content of the extract to an inositol compound with fewer phosphates.

Phytic acid may be thought of as inositol (“I” herein) hexaphosphate (IP₆). Inositol compound with fewer phosphates are such as IP₅, IP₄ and IP₃, the letter P indicating a phosphate group. The extract inherently has inositol (I) in it. These various compounds are of interest to this invention.

As an additional but optional feature of the invention, should only inositol be desired, and not its phosphates, then a phytase enzyme may be added after the above process, to remove any remaining phosphate groups.

The above and other features of this invention will be fully understood from the following detailed description.

### DETAILED DESCRIPTION OF THE INVENTION

The mesocarp, largely due to its unique biological progression through maturation to senescence, has a unique fruit (hull) composition of saccharides, polyhydroxy molecules inositol and sorbitol, and inositol phosphates, including phytic acid (myo inositol (1, 2, 3, 4, 5, 6) hexakisphosphate, as sodium, potassium, calcium or magnesium salts. In abbreviated form, phytic acid is referred to as IP₆). The flavonoids include anthocyanins, anthoxanthins (flavonols, flavones, flavanols, isoflavones), and polyphenols further polymerized to plant tannin structure with molecular weight greater than 800. Lignans are also present.

Plants, yeasts and fungi have enzymes which can synthesize inositol de novo, and also can synthesize inositol phosphates, inositol polyphosphates, inositol pyrophosphates, and further, produce inositol phosphates via dephosphorylation of phytic acid. The stereochemical array of inositol phosphates produced differ somewhat between plant and yeast/fungi/mammalian enzyme system. The inositol phosphates have recently been shown to have important physiological roles, and may have useful pharmacological activity.

Inositol phosphates so far identified in plants and animal include, in addition to phytic acid IP₆, I(1,4)P₂, I(4,5)P₂, I(1,4,5)P₃, I(1,3,4)P₃, I(1,2,3,4)P₄, I(1,3,4,6)P₄, I(1,3,4,5)P₄, I(1,4,5,6)P₄, I(3,4,5,6)P₄ and I(1,3,4,5,6)P₅. Plant enzymes apparently can produce I(1,4,5,6)P₄ and I(1,2,3,4)P₄, whereas I(1,3,4,5)P₄ is found in other eukaryotes. The inositol pyrophosphates, which contain di-phosphate ester linkages, have so far been shown to occur as IP₄-pyrophosphate, IP₅-pyrophosphate, and IP₄-pyrophosphate₂.

It is the purpose of this invention to utilize enzyme systems from both plants and other eukaryotic organisms to: (1) generate, and recover, a uniquely broad spectrum of all possible inositol phosphates, inositol polyphosphates, and inositol pyrophosphates, and (2) recover, in concentrated form, the flavonoids, phenolic acids, polyphenolics, terpenes, and polyhydroxy molecules contained in the almond hull. This is achieved by combining yeast/fungal/bacterial “fermentations” with plant enzyme reactions on a substrate of almond fruit extract.

In one procedure, almond fruit juice is extracted from comminuted dry mesocarp via aqueous counter current extraction to produce an aqueous extract. This extract will be used as the growth medium substrate for a 12-48 hour yeast batch “fermentation”, using a yeast with minimal to none extracellular phytase activity. This fermentation will effectively dispose of all of the fermentable sugars in the juice, namely fructose, glucose, and sucrose, leaving behind all of the inositol, sorbitol, most or all of the phytic acid, and of the other organics (flavonoids).

The fermentable sugars represent the majority of the compounds in the extracted juice, and the fermentation therefore represents an economical procedure for biological removal of those compounds, leaving behind a less complex solution of the molecules of interest. At the end of the fermentation, the yeast, and other solids, will be removed from the suspension using either centrifugation, or membrane filtration utilizing both micron pore size and high molecular weight (nominal) cutoff pore sized membranes.

Phytic acid, and other inositol phosphates will be separated from inositol, sorbitol, and other organics using preparative chromatographic methodology with anion exchange resins which selectively bind the highly charged phytic acid and inositol phosphates.

The polyhydroxy compounds inositol and sorbitol are then separated from the other organics with selective preparative chromatographic methods, or fractional crystallization techniques.

The three streams of dilute products (1) phytic acid and inositol phosphates (2) inositol and sorbitol (3) organics including flavonoids, are then each concentrated to final product specifications, or dried and/or crystallized to final product specifications. Product categories (1), (2) and (3) can be further separated using supercritical carbon dioxide techniques, or preparative chromatographic techniques, or fractional crystallization techniques, to pure single component products.

In other procedures, whose objectives are to produce more of the inositol phosphates, fermentations will be run from 12 to 48 hours in a batch mode, and then can be carried further for specified periods of time (20 to 200 hours) in fed batch aerobic fermentation protocols which will allow time for the several phosphatase (such as phytase) enzymes, and kinase enzymes, to produce the inositol phosphates.

The enzymes will be present in the extracted juice of the almond mesocarp, plus those which can be added with amounts of mesoderm “mash” (i.e. a slurry of suspended almond mesoderm tissue), and also can as an optimal feature be present as extracellular enzyme activity supplied by selected fungi, such as Aspergillus ficuum, Aspergillus flavus, Aspergillus niger; selected bacterial strains, such as subtilis, pseudomonas, klebsiella; or selected yeast strains from amongst D. cerevisiae, S. pombe, Schwanniomyces castellii, S. boulardii. Additional supplemental phytase activity can be utilized from amongst several commercially available purified enzymes, such as IUPAC EC 3.1.38, and EC 3.1.3.26.

At the end of the longer term fed batch fermentations, the fermentation and enzyme activity will be stopped via short high temperature excursion at pasteurization temperatures such that enzymes and organisms are inactivated. The organisms will be separated from the fermentation suspension via centrifugation or filtration plus ultrafiltration. The varying combinations of inositol phosphates produced at each of the separately timed fermentation procedures will be collected through use of preparative chromatographic procedures, and either concentrated to a syrup, or dried to powders, or crystallized via fractional crystallization techniques.

The solution remaining after removal of inositol phosphates will contain the flavonoids, lignans, and will likewise be concentrated to a syrup, or dried to powders. Individual components of the families of flavonoids, lignans, can be separated from the mixture using preparative chromatographic techniques.

### EXAMPLE 1

Almond fruit juice extract at between 10 degree Brix-35 degree Brix is used as growth and fermentation medium for one of the yeasts, S. cerevisiae, S. pombe, S. boulardii, Schwanniomyces castellii. A batch fermentation procedure, with aeration and stirring, at 30 degree C. is carried out for between about 5 to about 20 hours. For all of the yeasts indicated, intrinsic phytase activity will reduce the phytic acid content on the juice extract to mixture of phytic acid, and inositol phosphates with one to five phosphate groups esterified to the molecule. Schwanniomyces castellii is the most active of these yeasts for this phytase action.

The batch fermentation eliminates the fermentable sugars in the juice, metabolized by the yeasts to increase total biomass and phytase activity, including fructose, glucose, sucrose, and especially for Schwanniomyces castellii, the oligosaccharides melibiose and importantly, galactinol, a disaccharide in almond fruit which contains inositol. Biological removal of the sugars provides a less complex solution from which to recover a mixture of phytic acid, inositol phosphates, inositol, and increased concentration, on a weight-to-weight basis, of the polyphenolics from the resulting solution at the end of the batch run.

At the end of the batch run, a short temperature excursion to pasteurization temperature, to inactivate the yeast, is performed, and then the yeasts are filtered from the batch solution, to be used for animal feed purposes.

Alternatively, the yeasts, especially S. boulardii, and Schwanniomyces castellii, can be filtered from the batch solution while still viable, and then used as a more beneficial probiotic for ruminant animals, monogastric animals, as well as for humans.

### EXAMPLE 2

Following the batch fermentation described in example 1, above, the batch suspension, now containing largely “spent” yeasts which are no longer fermenting and reproducing, although still viable, and phytic acid, inositol, and inositol phosphates, can then be used as the reaction substrate for added, purified, phytase enzymes, such as IUPAC EC 3.1.38, and EC 3.1.3.26. This is now an enzyme reactor, as distinct from a batch “fermentation” process, in which the enzymes now complete the process of dephosphorylation of the inositol phosphates, yielding a solution with increased pure inositol concentration, no phytic acid or inositol phosphates, and increased polyphenolics concentration. Dependent upon the particular phytase enzymes employed in this step, temperature and pH are adjusted to optimal conditions for the enzyme action.

This invention is not to be limited by the embodiments shown in the drawing and described in the description, which are given by way of example and not of limitation, but only in accordance with the scope of the appended claims.

I claim:

1. A process to enhance the production of inositol, inositol phosphates and other compounds from almond hulls, said process comprising:
a. subjecting almond hulls to water extraction, thereafter separating the fibrous remnants of the almond hulls to produce a water extract containing the water soluble constituents of the almond hulls, including inositol, phytic acid, and sugars;
b. adding to said water extract a yeast of the type which consumes said sugars and reduces at least some of the phytic acid to an inositol phosphate with fewer than six phosphate groups while producing increased yeast biomass;
c. separating said yeast solids to produce an aqueous solution of inositol, inositol phosphates, and other soluble constituents of the water extract.

2. A process according to claim 1 in which said fermentation of step b proceeds for between about 5 and about 20 hours.

3. A process according to claim 1 in which said solution produced in step c has added to it to an enzyme of the type which dephosphorylates phytic acid and inositol phosphates to produce inositol.

4. A process according to claim 1 in which said yeast is selected from the group consisting of S. cerevisiae, S. pombe, S. boulardii, Schwanniomyces castellii, and combinations thereof.

5. A process according to claim 3 in which said enzyme is selected from the group consisting of IUPAC EC 3.1.38, and EC 3.1.3.26, and combinations thereof.

6. The product produced by the process of claim 1.
